(19) United States
(12) Patent Application Publication
(10) Pub. No.: US 2026/0360563 A1
(43) Pub. Date: Nov. 12, 2026

(54) INTELLIGENT TISSUE CLASSIFIER OF BONE AND SOFT TISSUE

(71) Applicant: AUSTRALIAN INSTITUTE OF ROBOTIC ORTHOPAEDICS PTY LTD., Nedlands (AU)

(72) Inventors: Riaz Jan Kjell Khan, Cottesloe (AU); Daniel Paul Fick, Cottesloe (AU); William Brett Robertson, North Fremantle (AU); Richard Chipper, Mount Claremont (WA); Joshua Goncalves, Atwell (AU)

(21) Appl. No.: 19/745,716

(22) Filed: Jul. 17, 2026

Related U.S. Application Data
(63) Continuation of application No. 17/415,193, filed on Dec. 19, 2019.

(30) Foreign Application Priority Data
Dec. 20, 2018 (AU) 2018904877

(51) Int. Cl. G16H 50/70; A61B 5/00; A61B 5/01; A61B 5/103; G16H 10/60

(52) U.S. Cl. CPC G16H 50/70; A61B 5/01; A61B 5/1032; A61B 5/4509; A61B 5/4514; A61B 5/4519; A61B 5/4533; A61B 5/4872; A61B 5/4875; A61B 5/7264; G16H 10/60; A61B 2562/0204

(57) ABSTRACT

A method for intraoperative state analysis and classification of articular tissue comprising the steps of: sensing data of the articular tissue via a plurality of sensors situated in proximity to the articular tissue for detecting a response to a stimulus; determining state analysis and classification information through a verified means; processing the sensed data into a form adapted to be evaluated against a comparator; generating a means of predicting the state analysis and classification utilizing a historical dataset of processed information; and performing the state analysis and classification using the generated means based on processing the stimulus response obtained intraoperatively.

### CROSS-REFERENCE TO RELATED APPLICATIONS

This application is a continuation of U.S. application Ser. No. 17/415,193, filed Jun. 17, 2021, which is a U.S. national phase of International Patent Application No. PCT/AU2019/051404 filed on Dec. 19, 2019, which claims priority to Australian Patent Application No. 2018904877, filed in Australia on Dec. 20, 2018. The entire contents of these applications are hereby incorporated herein by this reference.

### TECHNICAL FIELD

The present invention relates generally to analysis of bone and soft tissue. More specifically, the invention relates to the biological state analysis and classification of said tissue within an intraoperative environment.

The invention has been developed primarily for use in methods and systems for orthopaedic surgery and, in particular to systems and methods for the biological state analysis and classification of bone and soft tissue in orthopaedic surgery applications including post-operative care and evaluation of orthopaedic surgical procedures and will be described hereinafter with reference to this application. However, it will be appreciated that the invention is not limited to this particular field of use.

### BACKGROUND OF THE INVENTION

Any discussion of the background art throughout the specification should in no way be considered as an admission that such background art is prior art, nor that such background art is widely known or forms part of the common general knowledge in the field in Australia or worldwide.

All references, including any patents or patent applications, cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art, in Australia or in any other country.

Understanding the state and classification of tissue allows various changes and precautions to be taken during surgery which can increase patient success and recovery rates.

This is especially apparent in surgeries involving the musculoskeletal system where poor tissue state can have physical implications on body movement and range, likely accompanied by a degree of pain. Articular tissue such as cartilage, muscle and bone comprise the joints within this system that allow it to function, with its performance naturally degrading as they do.

Total knee arthroplasty is a prominent form of orthopaedic surgery where a predefined amount of hard tissue must be removed from bones participating in the knee joint using an osteotomy. Prosthetic implants are then fixated to the remaining bone to replace that which was removed. This surgery is typically required when the cartilage surrounding the femur, tibia and patella bones start to dissipate. This causes them to grind against each other during normal movement and withstand increased levels of stress that would normally have been absorbed by the cartilage. By inserting a prosthetic implant into these bones, which is designed to absorb stress in place of them, these effects can be significantly reduced.

A prosthetic implant can be fixated to the articular hard tissue using one of two different methods. The first is by attaching it directly to the hard tissue and relying on osseointegration, which refers to the hard tissue naturally growing into the prosthetic implant and stabilising it. This is the more natural method and generally tends to result in a stronger connective interface. The second method is through bone cement which is a fixative that forms a strong bond between the prosthetic implant and hard tissue. This method relies on the strength and maintainability of the cement, which is not as reliable as natural bone and can potentially be toxic depending on its production. It also leads to increased difficulty when positioning osteotomies and inserting the prosthetic implant as the size of the applied cement and its distribution before and after pressure is applied must be taken into account. The implant fixation method is generally selected based on the health of the underlying tissue. If it is healthy, then direct implantation is possible, otherwise reliance must be placed on bone cement.

According to the national centre for health statistics, in the US alone, over 700,000 total knee arthroplasty operations are performed annually which is expected to rise to 3.48 million by the year 2030. The vast majority of these operations are initially successful, with patients, whose mean age is 66.2 years old, reporting significantly less pain and increased mobility. However, after a duration has passed postoperatively, it is possible that issues may arise which require additional revision surgery. This is the case for around 8% of all knee replacement procedures, with this number set to similarly increase by 601% in 2030.

These issues are related to the quality of the interface between the prosthetic implant and the hard tissue. They are generally caused by an imperfect implantation operation, although continual or abrupt trauma can also result in or at least exacerbate them. They include aseptic loosening, infection, polyethylene wear, instability, pain, osteolysis and malposition which are responsible for 23.1%, 18.4%, 18.1%, 17.7%, 9.3%, 4.5% and 2.9% of all revision surgeries respectively. Each is generally interdependent, with the appearance of one potentially being triggered or influenced by the beginnings of another.

Aseptic loosening is the largest cause of revision surgery and refers to the fixation failure at the implant and articular tissue interface, leading to increasing levels of pain and instability for the patient. The aetiology of aseptic loosening comprises of four main causes. The first is a biological response to wear particles from the prosthetic implant. Provided that enough stress is applied, it is possible that small particles within the critical range of 0.3 to 10 micrometres may detach from the implant. Depending on the health of the articular tissue and the genetics of the patient, this may then cause a macrophage-based inflammatory response, leading to osteolysis.

The second cause is a build-up of intra-articular fluid pressure. This is a result of the overproduction of synovial fluid due to exposed hard tissue or wear particles surrounding the joint. This excess of synovial fluid creates additional pressure which may result in abnormal bone perfusion or ischemia, leading to necrosis and osteolysis.

The third cause is the physical design of the implant wherein the pattern and profile of the surface influence the rate and potential of osseointegration. If this influence is negative, then the amount of ingrowth may not be enough to stabilise and fixate the prosthetic.

The fourth cause is the biological specifics of the patient which includes characteristics specific to them such as their age and habits, any pre-existing infections or diseases that may affect the joint, and their genetics. If the patient partakes in routine physical exercise, such as running, or naturally has weak joints, then their risk will increase.

This causation can be at least partially attributed to the state or alternatively the health of the underlying articular tissue and the corresponding patient. Although various procedures that are capable of analysing the state and structure of tissue exist, finalised conclusions cannot be made until the state of the tissue is established intraoperatively.

Common methods of intraoperative tissue state analysis and classification are generally reliant on the visual analysis of the surgeon administering the operation. This means that any adjustments or changes based on the tissue state independent of preoperative knowledge is typically reliant on the experience of the surgeon. This dependence leads to variance in patient satisfaction and operation longevity, with a reported 20% of total knee arthroplasty patients being unsatisfied with their results. By gaining increased knowledge of the subject tissue intraoperatively, the quality of surgical operations and the surgeons' ability to react to unknown circumstances can increase.

Preoperative approaches to achieving some degree of knowledge surrounding tissue state and classification are typically based around invasive procedures that require sections of tissue or cells to be removed for subsequent analysis. Depending on the analysis technique, information such as the composition, topology, mineral and water content for any particular tissue sample can be discerned.

The preparation required for these collected tissue samples can be lengthy and includes numerous preservation and fixation techniques in an attempt to maintain the original, natural state of the tissue against the apparent degradation. Despite this, the composition of the tissue will generally differ from its original composition to varying extents depending on this preparation process. This disparity must be appropriately accounted for in any subsequent analysis to ensure that the results are portable and comparable.

Although the patient is usually under the influence of anaesthesia during these invasive procedures, some will still prove painful depending on how easily the tissue surrounding the extraction point can be numbed. Recovery time will also be required depending on the procedure, which will likely mean mild discomfort for the patient. For their body, this will mean the regeneration of extracted tissue, resulting in temporary vulnerability, including the risk of infection. These occurrences, or more-so consequences, limit the potential for tissue retrieval as only minute amounts can be taken for fear of patient distress.

The small amount of tissue or cells removed is therefore responsible for representing their surrounding population which may not necessarily be true in all cases. If this is not the case, then incorrect conclusions may be observed which would lead to problems larger than potential retesting.

Approaches that do not require invasive procedures or direct extraction of tissue do exist but are focused on the structural analysis of tissue as opposed to its composition. This can assist in classification as the location is useful in defining the type of articular tissue, although it has comparably little function for determining the state.

Non-invasive procedures generally work by exporting energy of some form into the subject and monitoring that energy when it either reflects back or passes through. While this is typically considered safe, the more popular procedures tend to revolve around the usage of X-rays, which is a form of radiation. The risks associated with this are decreased through tight control over the machinery involved in their output, but still exist nonetheless.

In these preoperative approaches, the analysis is mainly performed by humans which means that the accuracy of the results and conclusions cannot be determinatively proven. This is because an individual's skill is largely dependent on their experience and education, which means that additional opinions are sometimes necessary.

With advancements in artificial intelligence and data science, the processing of large amounts of data into meaningful information, which is otherwise difficult to discern, is now possible. This allows new technologies, such as optical and acoustic analysis, to provide further information relating to the state and classification of tissue whilst lacking many of the drawbacks experienced by current techniques.

The invention herein disclosed provides a method of performing state analysis and classification of articular tissue within an intraoperative environment.

### SUMMARY

The invention provides systems and methods for articular tissue state analysis and classification that can be performed intraoperatively. In particular, the invention provides methods for the collection of data from different sensors, the processing of that data and its inclusion in preparatory and executory phases of machine learning, data science and mathematical algorithms and techniques.

In one aspect, the invention provides systems for gathering data of differing types which may describe various attributes relating to the articular tissue based on the method of data generation. A preferred system comprises multiple different sensors and possible capture tools within a typical surgical environment where the sensors operate collectively in an automated fashion to assist the surgeon transparently.

The selection of sensors may comprise at least one sensor which may exist independently or as part of a sensor system or set of sensors. Individual sensors may be able to monitor, sense and collect data based on various attributes, characteristics, occurrences or measures from different angles, positions, vicinities, placements or arrangements that exist with, within or are directed at their subject which may be articular tissue, the surrounding environment, the result of an action or interaction, individual or collections of systems or devices and any other advantageous source or series of sources therewith. The sensors may be completely self-contained or may require additional devices, services, platforms, or conditions to interface with, configure or operate appropriately.

Selected sensors may include those based on Raman spectroscopy, spectral imaging, hyperspectral imaging, optical imaging, thermal imaging, fluorescence spectroscopy, microscopy, acoustics, 3D metrology, optical coherence tomography, movement, balance, laser power and any other singular, combination or sequence of sensing forms which can contribute data intraoperatively.

Sensed attributes may include tissue composition, hydration, density, necrosis, colouration, reflectance, heat consistency and any other singular, combination or sequence of attributes that could be used to describe state or class.

In one embodiment, sensing may occur transparently within the surgery as the surgeon is performing at least one action of work. This sensing process may pause, omit or otherwise disregard sensed data in the scenario that any obstructions occur that result in changes to viable sensing conditions which may include personnel obscuring the sensors, an abundance of light or noise, unfavourable movement of the subject, and any other singular, combination or sequence of adverse sensing conditions.

In another embodiment, sensing may interrupt the natural progression of a surgery for a predetermined or intraoperatively calculated duration to provide an environment which is beneficial to the sensing procedure. This interruption may comprise changes to the surgical environment which may include the temporary removal of personnel, lighting modifications or dimming, atmospheric alterations, subject repositioning and any other singular, combination or sequence of environmental changes.

In one embodiment, sensed data may be interpreted based on the individual sensor that provided it, irrespective to other sensors which may surround or operate in relation to it.

In another embodiment, sensed data may be interpreted based on a system or set of or multiple sets of sensors where inclusion may be coincidental or defined by attributes, similarities, conditions, states and any other singular, combination or sequence of grouping factors.

In another embodiment, sensed data interpreted based on collections of sensors may be summarised to provide increasingly accurate information, be used as a fault tolerance measure to determine working efficiency or in any other singular, combination or sequence of ways in which the unison of the involved sensors may be beneficial.

In a further embodiment, the interpretation of sensed data originating from a singular, system, set of or multiple sets of sensors may occur irrespective to or with respect to environmental or internal conditions and physical sensor arrangement which may include temperature, humidity, pressure, varying amounts of lighting and their directions, different positions, angles, vicinities or placements and any other singular, combination or sequence of influential factors.

In another aspect, the invention provides methods for pre-processing sensed data into at least one different, sequential form that may increase its usability. A preferred method comprises cleaning the data to remove noise and redundancy, changing the format or arrangement of the data and sampling the data to segregate portions or areas which may be deemed beneficial.

Sensed data will be provided by the selection of sensors whose form will be dependent on these sensors and may include wavelengths, signals, arbitrary numbers, equations, coordinates, models or any other singular, combination or sequence of directly or indirectly interpretable data forms.

The pre-processing of sensed data may involve various different, similar or identical methods in the same or alternating orders to produce single or multiple sequential forms leading up to a finalised form. Some algorithms or methods may not be available for all forms of data or sensor types, although this may change provided the appropriate modifications. Each individual form may produce benefit with the intent of the sequential transformation not necessarily being to result in the final form alone.

In one embodiment, large sections or collections of data whose additional value may not have a significant impact on conclusions derived from the remaining data or which are incorrect or erroneous may be removed. This may include data in which the event of interest or aspects that may support it does not occur, that consists of erratic values, or any other singular, combination or sequence of states in which the extraction of utility is impractical or negligible. For example, acoustic samples may be trimmed to remove the portions that are silent.

In another embodiment, sets or series of data corresponding to the same, similar or different events which bear structural similarities may be averaged or otherwise combined to outline portions or areas where variations including noise or erroneous data may exist, which can then be removed from the either singular or combined sets or series of data.

In a further embodiment, similar data which may have little value individually may be summarised or combined into single or multiple representative sets of data to decrease the sheer amount of data without significantly impacting any derived conclusions.

In one embodiment, the format, form or structure of the data may be rearranged, altered or changed to produce additional or alternate utility through methods which may include flattening the data and changing the positions or relationships between specific or ordered values.

In one embodiment, the data may be sampled to extract areas or portions deemed more advantageous or to create a series or set of data samples that can be processed or manipulated separately for purposes such as cross validation and testing.

In another aspect, the invention provides methods for manipulating pre-processed data into at least one different, sequential form that may increase its evaluability. A preferred method comprises normalising the pre-processed data to restrict it within a comparable range, decomposing the data to define its constituent elements, performing fractional abundance estimations of these elements and aggregating them into an entity of prominent utility.

The manipulation of pre-processed data may involve various different, similar or identical methods in the same or alternating orders to produce single or multiple sequential forms leading up to a finalised form. Some algorithms or methods may not be available for all forms of data or sensor types, although this may change provided the appropriate modifications. Each individual form may produce benefit with the intent of the sequential transformation not necessarily being to result in the final form alone.

Constituent elements represent independent or summarised components that exist within the original data. The number and type of constituent elements produced is dependent on the data form, any previously executed processing methods, the situation or environment in which the sensing occurred and any other singular, combination or sequence of conditions which may result in multiple components existing within the data. Constituent elements of articular tissue will typically comprise cancellous bone, cortical bone, cartilage, fat, ligament, muscle or meniscus. Additional constituent elements may exist as specialisations of these which may include composition, hydration, density, necrosis, reflectance, temperature, and any other singular, combination or sequence of elements that could possibly describe the state of articular tissue.

Fractional abundances represent the presence of a specific singular or combination of constituent elements within the data. This abundance or relative amount depends on the methods, situation, environment and any other singular, combination or sequence of conditions that lead to or influence the extraction of their associated constituent elements.

In one embodiment, the data may be normalised through algorithms and methods such as constant shifts, smoothing, scaling, standard normal variate, baseline correction, continuum removal or any other singular, combination or sequence of algorithms and methods capable of improving data consistency.

In one embodiment, the data may be decomposed or deconvoluted into its constituent elements or features which may be achieved through algorithms and methods including the automatic target generation process, pixel purity index, N-FINDR, matched filtering, independent component analysis, non-linear least squares, fuzzy k-means and any other singular, combination or sequence of algorithms and methods capable of decomposition. Some of these methods or algorithms may not be possible without potential modification depending on the form of the supplied data and its purpose.

In another embodiment, constituent elements may be identified prior to beginning their extraction to determine which may exist within the data and any directives that may assist in their extraction.

In another embodiment, a series or set of constituent elements may be averaged or combined in a beneficial manner provided they share or do not share any similar patterns or other elements which can be used as a means of grouping. This may occur when the number of constituent elements is greater than the expected number.

In a further embodiment, decomposition may comprise the reduction of data dimensions to reduce complexity and computational load which may be performed through algorithms and methods including decision trees, random forests, high correlation filters, backward feature elimination, factor analysis, principal component analysis, linear discriminant analysis, generalised discriminant analysis and any other singular, combination or sequence of algorithms and methods capable of removing dimensions or otherwise assisting in this process.

In one embodiment, fractional abundance estimations may be calculated through algorithms and methods such as unconstrained least squares, non-negative constrained least squares, fully constrained least squares, fuzzy k-means, and any other singular, combination or sequence of algorithms or methods capable of estimating fractional abundance or otherwise assisting in this process.

In another embodiment, portions or areas of fractional abundance estimations that are advantageous, beneficial or provide alternate utility may be segregated.

In a further embodiment, this segregation may be focused on an area where the series or set of fractional abundances differ to a large or meaningful degree which may provide benefit or additional utility to subsequent machine learning, data science and mathematical algorithms or methods.

In one embodiment, the representative, constituent or otherwise singular sets of data, elements or features may be aggregated together into a single entity or into fewer entities that can be processed more easily whilst maintaining similar or increased utility.

In another aspect, the invention provides methods for preparatory and executory phases of machine learning, data science and mathematical algorithms and methods. A preferred method comprises using verified processed data to train multiple machine learning, data science and mathematical algorithms or methods in a supervised manner which can then be provided with raw processed data to generate corresponding output based on this training.

The data used may be processed to ensure that it is in a form which promotes increased utility when used in conjunction with a machine learning, data science or mathematical algorithm or method. This form may be dependent on the initial state of the sensed data, the purposed algorithm or method and the intent behind its usage. It may be defined as normalised, cleaned data that contains only the processed or representative portions of the original data set that would be beneficial to a particular machine learning, data science or mathematical algorithm or method as defined within the previous aspects.

Some processed data may be verified such that its intended output depending on the machine learning, data science or mathematical algorithm or method has already been determined and confirmed through external means.

Machine learning, data science and mathematical algorithms or methods may be based around supervised approaches. These algorithms or methods may include linear and polynomial regression, logistic regression, naïve bayesian networks, bayesian networks, support vector machines, decision trees, random forests, k-nearest neighbour classifiers, neural networks and any other singular, combination or sequence of supervised approaches.

In one embodiment, the verified processed data pool will be split into at least two divisions of differing purposes where this split is not necessarily even or proportional.

In another embodiment, a single or set of split verified processed data may be provided into a single or multiple machine learning, data science or mathematical algorithms or methods in a sequential, simultaneous or periodic manner.

In one embodiment, a single or set of all or a portion of the remaining split verified processed data may be provided to previously trained single or multiple machine learning, data science or mathematical algorithms or methods to gauge the accuracy of their corresponding output against the externally confirmed output.

In another embodiment, the accuracy of a particular trained machine learning, data science or mathematical algorithm or method may be deemed sufficient depending on its statistical significance which may be determined or influenced by the application of its predictions or estimations.

In a further embodiment, if the accuracy has not proven to be sufficient, then the verified data selected, its input procedure, the single or multiple machine learning, data science or mathematical algorithms or methods or any other singular, combination or sequence of causation may be modified, removed, rearranged or added to possibly result in increased accuracy.

In one embodiment, raw processed data may be provided to a single or set of trained machine learning, data science or mathematical algorithms or methods to produce corresponding output.

In another embodiment, corresponding output from at least two machine learning, data science or mathematical algorithms or methods may be averaged, combined or compared to possibly reach increasingly definitive conclusions.

According to an aspect of the invention, there is provided a method for intraoperative state analysis and classification of articular tissue comprising the steps of:
sensing data of the articular tissue via a plurality of sensors situated in proximity to the articular tissue; determining state analysis and classification information through verified means; processing sensed data into a form adapted to be evaluated against a comparator;
generating a means of predicting state analysis and classification utilising the historical dataset of processed information; and
performing state analysis and classification using the generated means based on processed stimulus provided intraoperatively.

The sensors may comprise at least one sensor which may exist independently or as part of a sensor system or set of sensors. The sensors may be completely self-contained or may require additional devices, services, conditions, platforms or any other singular, combination or sequence of requirements to be interfaced with, configured or operated appropriately. Individual sensors may be able to monitor, sense and collect data based on various properties, characteristics, occurrences, measures or any other singular, combination or sequence of features. This may be done from different angles, positions, proximities, vicinities, movements, speeds, placements, arrangements or any other singular, combination or sequence of setups that exist with, within or are directed by their subject.

The subject may be articular tissue, the surrounding environment, the result of an action or interaction, individual or collections of systems or devices and any other advantageous source or series of sources therewith. The subject may be treated through any possible method in order to affect its original, initial or current state for the purpose of preservation, identification, unification, fixation or any other singular, combination or sequence of status altercations. The subject may be modified structurally, chemically or through any other singular, combination or sequence of approaches which may change its form as part of or independent to any intraoperative procedures, surgeries or any other singular, combination or sequence of medical operations.

The sensors may operate to provide data based on each individual sensor or sensor type, groupings of sensors or sensor types, positioning of sensors or sensor types, angles of sensors or sensor types, vicinities of sensors or sensor types or any other singular, combination or sequence of configurations or arrangements which may provide advantageous combinations of data that may be independently or collectively analysed. The sensors may work in an automated fashion, through manual triggering or through any combination or sequence of the two. Each sensor may not necessarily have the same control scheme, with any number of control schemes being applicable to any number of sensors.

Manual triggering involves any form of trigger that is not automated which may include buttons, voice commands, gesture controls, all forms of physical actuation and any other singular, combination or sequence of manual controls.

The sensors may engage in sensing indefinitely, periodically, singularly or in any other singular, combination or sequence of sensing approaches which may be influenced by the situation, environment, user control, sensor configuration and any other singular, combination or sequence of variables capable of having a direct or indirect effect.

The sensing may work in real-time, near real-time, through some form of delayed processing or in any other singular, combination or sequence of processing approaches which may be influenced by the situation, environment, user control, sensor configuration and any other singular, combination or sequence of variables capable of having a direct or indirect effect.

The sensor may need external involvement to operate correctly which may include changes to its position, angle, vicinity, proximity, configuration, lighting, timing or any other singular, combination or sequence of sensor, situational or environment changes.

Data or other information may be provided by verified personnel, databases, systems or any other singular, combination or sequence of internal or external sources which may have been verified and validated.

State analysis refers to the state of articular tissue comprising composition, hydration, density, necrosis, colouration, reflectance, heat consistency and any other properties or characteristics that could be used to describe state.

Classification refers to the type of articular tissue amongst the possible types which may include cancellous bone, cortical bone, cartilage, fat, ligament, muscle, meniscus and any other articular tissue type.

Verified means may comprise an expert surgical declaration, a single or multiple postoperative verification techniques and any other singular, combination or sequences of forms of verification that can be deemed to have an appropriate level of accuracy. Verification may be supported by medical records and history, family lineage, genetic factors, the typical lifestyle and environment of the subject and any other singular, combination or sequence of factors to which conclusions may not be directly derivable but of which can be used to support those that are.

The level of accuracy appropriate for any particular verification may depend on the technique used, the subject that it was used on, the personnel applying the technique, the environment or situation where the technique occurs, the information being derived from the technique, the amount of times the technique is repeated, the amount of information or data supporting the technique, the amount of similar or different techniques that are used in unison and any other singular, combination or sequence of influential elements.

The processing of sensed data involves at least one action of work relating to the transformation of said sensed data into an evaluable form. The transformation of data may comprise at least one action of work involving a single, multiple, combination or sequence of pre-processing steps.

A pre-processing step may comprise cleaning the data including the removal or fixing of any noisy, erroneous or redundant data and any other singular, combination or sequence of processes which may remove negligent data or increase the overall utility of the remaining data. A pre-processing step may comprise formatting the data including the rearrangement of data into a more appropriate structure or form, the flattening of data or extraction from its current storage and any other singular, combination or sequence of formatting which may increase the usability of the data. A pre-processing step may comprise sampling the data including the selection or division of portions of said data or any other singular, combination or sequence of processes which may result in more representative or advantageous data.

The transformation of data may comprise at least one action of work involving a single, multiple, combination or sequence of raw or pre-processed data manipulations. The manipulation of raw or pre-processed data may include the normalisation, scaling or alignment of said data so that it may exist within a comparable range or achieve some additional level of comparability. The manipulation of raw or pre-processed data may include the decomposition or deconvolution of said data so that representative or otherwise specific features or portions of data can be split into constituent elements or elements which provide more utility individually. The manipulation of said raw or pre-processed data may comprise the estimation of fractional abundances based on the constituent elements identified within the deconvoluted data. The manipulation of said raw or pre-processed data may include the aggregation of said data so that individual features, constituent elements, sections or portions of data may be combined into a single entity.

The transformation of data may comprise at least one action of work relating to any other singular, combination or sequence of processes, manipulations, generations, alterations or any other functions, techniques or approaches that may better prepare said data for usage.

The processing of sensed data may not be required or may be partially performed if an additional entity such as a sensor controller or bridging device has performed this processing individually or independently.

The ordering or existence of data pre-processing and manipulation processes may not reflect their ordering herein.

The comparator is a set of data in a similar or otherwise comparable form belonging to a singular, combination or sequence of articular tissue.

The generation of a means of performing tissue state analysis and classification may comprise at least one action of work involving the training or construction of a machine learning, data science, mathematical or computational entity, concept, model, equation or any other singular, combination or sequence of embodiments.

Training refers to the provision of data to a machine learning, data science, mathematical or computational entity, concept, model, equation or any other singular, combination or sequence of embodiments where said data may create, populate, change, augment, calculate or otherwise participate in any preliminary or preparatory phases required to establish the functionality of the embodiment.

The historical dataset of processed data may comprise data which has been previously processed at any point prior by any singular, combination or sequence of means compatible with and of which can be used within the generation of predictive means.

Data existing within the historical dataset may originate from the single, multiple, collection or sequence of sensors used, may be provided externally through personnel of verifiable expertise, previous medical records and history, family lineage, patent lifestyle and intended environment, may be retrieved from accurate and well-tested data sources, prior discovered connections and any other singular, combination or sequence of sources from any time period that may provide usable or supporting data.

Construction refers to at least one action of work involving the development of a simulation or other entity which may be used in the prediction, generation, calculation, verification, validation or any other singular, combination or sequence of usages relating to a machine learning, data science, mathematical or computational entity, concept, model, equation or any other singular, combination or sequence of embodiments.

A trained machine learning, data science, mathematical or computational entity, concept, model, equation or any other singular, combination or sequence of embodiments may predict tissue state, tissue classification or both to individually varying degrees of accuracy either independently, in collaboration with other embodiments, as part of a set or through any other form of grouping or cooperative setup.

The method for intraoperative articular tissue state analysis and classification comprises spectral or hyperspectral analysis.

The spectral or hyperspectral sensors may include any singular or combination of possible bands available within the entire electromagnetic spectrum.

Additional light sources may be involved to augment or adjust the spectral or hyperspectral analysis which may include UV, visible, infrared and any other singular, combination or sequence of light sources.

Pre-processing steps may comprise the removal of any noisy, redundant or erroneous data, the rearrangement or formatting of data into a more appropriate structure or form, the adjustment or normalisation of data, the sampling of data to extract more representative or beneficial portions or sections, and any other singular, combination or sequence of steps which may provide beneficial or alternate utility.

The manipulation of raw or pre-processed spectral data may comprise at least one action of work relating to the decomposition of said spectral data.

The decomposition of spectral data may include dimensionality reduction which may involve techniques such as principal component analysis, linear discriminant analysis, quadratic discriminant analysis and any other singular, combination or sequence of techniques which can reduce dimensionality.

The decomposition of spectral data may comprise at least one action of work relating to the deconvolution, endmember determination or disintegration of various spectrums within said spectral data into several spectral signatures or other form of identifiable data belonging to individual or groups of endmembers to which the original spectral data consists of.

The decomposition of spectral data may comprise at least one action of work relating to the inversion of deconvoluted spectral signatures and their original spectrums to estimate the fractional abundances of each endmember within the spectrum or spectral data.

The manipulation of raw or pre-processed spectral data may comprise at least one action of work relating to outlining sections, areas or portions of the spectral data or derivatives in which various wavelengths, measurements or other information differ from each other to a degree that is deemed beneficial.

The method for intraoperative articular tissue state analysis and classification comprises acoustic analysis. Acoustic sensors may have any possible sampling rate including those within the human conceivable, ultrasonic and any other singular, combination or sequence of ranges. Acoustic signals may be produced by any object or actuator capable of creating a sound independently, through contact with another entity, or through any other singular, combination or sequence of actions or functions.

Pre-processing steps may comprise the removal of any noisy, redundant or erroneous data, the rearrangement or formatting of data into a more appropriate structure or form, the adjustment or normalisation of data, the sampling of data to extract more representative or beneficial portions or sections, and any other singular, combination or sequence of steps which may provide beneficial or alternate utility.

The manipulation of raw or pre-processed acoustic data may comprise at least one action of work relating to the decomposition of said acoustic data.

The decomposition of acoustic data may include dimensionality reduction which may involve techniques such as principal component analysis, linear discriminant analysis, quadratic discriminant analysis and any other singular, combination or sequence of techniques which can reduce dimensionality.

The decomposition of acoustic data may comprise at least one action of work relating to the deconvolution, endmember determination or disintegration of an acoustic signal or data into several sets of frequencies, amplitudes or any other singular, combination or sequence of identifiable aspects belonging to individual or groups of sound sources to which the original acoustic data consists of.

The decomposition of acoustic data may comprise at least one action of work relating to the inversion of said disintegrated acoustic signals and their original signals to estimate the fractional abundances of each endmember within the acoustic signals or data.

The manipulation of raw or pre-processed acoustic data may comprise at least one action of work relating to outlining sections, areas or portions of said acoustic data or derivatives in which various frequencies, amplitudes, measurements or other information differ from each other to a degree that is deemed beneficial.

Performing state analysis and classification may involve the execution of a previously generated or constructed means of prediction and may involve at least one action of work related to their preparation or initialisation.

A previously generated means may comprise a machine learning, data science, mathematical or computational entity, concept, model, equation or any other singular, combination or sequence of embodiments.

Performing state analysis and classification may involve the execution of at least one action of work relating to the usage of at least one simulation or other entity which may be used in prediction, generation, calculation, verification, validation or any other singular, combination or sequence of usages relating or as assistance to a machine learning, data science, mathematical or computational entity, concept, model, equation or any other singular, combination or sequence of embodiments.

The sensed, raw, pre-processed, manipulated, interpreted, processed, usable, evaluable or any other singular, combination or sequence of generated, derived or received data may be stored electronically which may be done offline, online or through some combination of the two for later retrieval, processing, or any other singular, combination or sequence of forms of usage.

Any processing or storage required may occur internally in a custom or generic system, externally on a centralised, decentralised or otherwise online entity or any other singular, combination or sequence of computational approaches.

At least one action of work may occur within an intraoperative environment. At least one action of work may occur in the same, different or alternating order and may produce the same, similar, or different finalised result. At least one action of work may occur in real-time, near real-time, through some form of delayed processing or any other singular, combination or sequence of processing approaches.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 is an abstract schematic flow diagram depicting the intraoperative tissue type and composition analysis process comprising the steps required for the complete implementation of the preferred embodiment;

FIG. 2 is a detailed schematic flow diagram depicting the data sources and related procedures involved in the collection and accessibility of data as introduced in the exemplary data collection step in FIG. 1;

FIG. 3 is a detailed schematic flow diagram depicting the pre-processing and manipulation required to transform data into a more evaluable form for further usage within predictive algorithms and methods as introduced in the exemplary data processing step in FIG. 1;

FIG. 4 is a detailed schematic flow diagram depicting the types of predictive algorithms or methods capable of producing information and properties relating to tissue type and composition based on existing processed data as introduced in the exemplary data interpretation step in FIG. 1;

FIG. 5 illustrates generally a basic sensing system and a sample intraoperative environment in which it may be used;

FIG. 5A illustrates generally a hyperspectral sensing system of the system in FIG. 5 which collects spectral data based on a subject;

FIG. 5B illustrates generally an acoustic sensing system of the system in FIG. 5 which collects acoustic data based on a subject; and

FIG. 6 shows a computing device on which the various embodiments described herein may be implemented in accordance with an embodiment of the present invention.

### DEFINITIONS

The following definitions are provided as general definitions and should in no way limit the scope of the present invention to those terms alone but are put forth for a better understanding of the following description.

Unless defined otherwise, all technical and scientific terms used herein have the same meaning as commonly understood by those of ordinary skill in the art to which the invention belongs. It will be further understood that terms used herein should be interpreted as having a meaning that is consistent with their meaning in the context of this specification and the relevant art and will not be interpreted in an idealized or overly formal sense unless expressly so defined herein. For the purposes of the present invention, additional terms are defined below.

Furthermore, all definitions, as defined and used herein, should be understood to control over dictionary definitions, definitions in documents incorporated by reference, and/or ordinary meanings of the defined terms unless there is doubt as to the meaning of a particular term, in which case the common dictionary definition and/or common usage of the term will prevail.

For the purposes of the present invention, the following terms are defined below.

The articles “a” and “an” are used herein to refer to one or to more than one (that is to at least one) of the grammatical object of the article. By way of example, “an element” refers to one element or more than one element.

The term “about” is used herein to refer to quantities that vary by as much as 30%, preferably by as much as 20%, and more preferably by as much as 10% to a reference quantity.

The use of the word ‘about’ to qualify a number is merely an express indication that the number is not to be construed as a precise value.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Any one of the terms “including” or “which includes” or “that includes” as used herein is also an open term that also means including at least the elements/features that follow the term, but not excluding others. Thus, “including” is synonymous with and means “comprising”.

In the claims, as well as in the summary above and the description below, all transitional phrases such as “comprising”, “including”, “carrying”, “having”, “containing”, “involving”, “holding”, “composed of”, and the like are to be understood to be open-ended, that is to mean “including but not limited to”. Only the transitional phrases “consisting of” and “consisting essentially of” alone shall be closed or semi-closed transitional phrases, respectively.

The term, “real-time”, for example “displaying real-time data”, refers to the display of the data without intentional delay, given the processing limitations of the system and the time required to accurately measure the data.

The term, “near-real-time”, for example “obtaining real-time or near-real-time data” refers to the obtaining of data either without intentional delay (“real-time”) or as close to real-time as practically possible (that is with a small, but minimal, amount of delay) whether intentional or not within the constraints and processing limitations of the system for obtaining and recording or transmitting the data.

Although any methods and materials similar or equivalent to those described herein can be used in the practice or testing of the present invention, preferred methods and materials are described. It will be appreciated that the methods, apparatus and systems described herein may be implemented in a variety of ways and for a variety of purposes. The description here is by way of example only.

As used herein, the term “exemplary” is used in the sense of providing examples, as opposed to indicating quality. That is, an “exemplary embodiment” is an embodiment provided as an example, as opposed to necessarily being an embodiment of exemplary quality and serving as a desirable model or representing the best of its kind.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a single or multiple computer readable storage mediums which may comprise computer memory, one or more floppy discs, compact discs, optical discs, magnetic tapes, flash memories, circuit configurations in Field Programmable Gate Arrays or other semiconductor devices and any other non-transitory or tangible storage media. These mediums may be encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that perform methods of the present invention when executed need not reside on a single computer or processor but may be distributed in a modular fashion amongst a number of different computers or processors to implement various aspects of the present invention.

Computer-executable instructions may exist in a variety of forms, such as program modules, and may be executed by a singular, combination or sequence of computers or other devices whose level of collaboration may differ to varying degrees. Program modules generally include routines, programs, objects, components, data structures and any other singular, combination or sequence of components, structures or executable entities that perform particular tasks, implement specific abstract data types or coordinate different processes. The functionality of a program module may typically be combined or distributed as desired in various embodiments.

Data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys the relationship between the fields. However, any suitable mechanism may be used to establish a relationship between information in fields of a data structure, including through the use of pointers, tags or other mechanisms that establish relationship between data elements.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or”, as used herein in the specification and in the claims, should be understood to mean “either or both” of the elements so conjoined such that elements may be conjunctively present in some cases and disjunctively present in other cases. Multiple elements listed with “and/or” should be construed in the same fashion, that is “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); and any other embodiments which may comprise unions, intersections or otherwise other combinations of associated elements.

As used herein in the specification and in the claims, “or” should be understood to have the same meaning as “and/or” as defined above. For example, when separating items in a list, “or” or “and/or” shall be interpreted as being inclusive, that is the inclusion of at least one, but also including more than one, of a number or list of elements, and, optionally, additional unlisted items. Only terms clearly indicated to the contrary, such as “only one of” or “exactly one of”, or, when used in the claims, “consisting of’ will refer to the inclusion of exactly one element of a number or list of elements. In general, the term “or” as used herein shall only be interpreted as indicating exclusive alternatives (that is “one or the other but not both”) when preceded by terms of exclusivity, such as “either”, “one of”, “only one of”, or “exactly one of”, “Consisting essentially of”, when used in the claims, shall have its ordinary meaning as used in the field of patent law.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B”, or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); and any other embodiments which may comprise differing combinations of inclusions and exclusions of A, B and any other associated elements.

For the purpose of this specification, where method steps are described in sequence, the sequence does not necessarily mean that the steps are to be carried out in chronological order in that sequence. The steps may be carried out in a completely alternate order which may mean that some occur simultaneously or are omitted entirely.

In addition, where features or aspects of the invention are described in terms of Markush groups, those skilled in the art will recognise that the invention is also thereby described in terms of any individual member or subgroup of members of the Markush group.

### DETAILED DESCRIPTION

The following detailed description is an exemplification of the invention and should not be limited in scope by the embodiments depicted nor should it be understood in any way as a restriction on the broad description of the invention as set out hereinbefore. These embodiments are described in sufficient detail to allow those skilled in the art to practise or exercise the invention. The precise shape, size and appearance of the components described or illustrated are not expected of nor required from the invention unless stated otherwise. It is to be understood that any utilisation, combination or structural, logical, electrical and mechanical changes, variations, augmentations or modifications to any of the mentioned or otherwise related embodiments may be made without departing from the scope of the invention. Similarly, any functionally equivalent products, compositions and methods will also remain within this scope along with all singular, combination and sequences of steps, features, structures, sequences, processes, combinations and compounds referred to or indicated within this description either singularly or collectively.

The entire disclosure of all documentation including patents, patent applications, journal articles, laboratory manuals, books, charts, repositories, and any other form of documentation or otherwise referenced resources cited herein is by no means an admission of prior art, prior or common knowledge required by those skilled in the art or any other connections or assumptions towards the invention unless mentioned otherwise.

Throughout this description, unless stated otherwise, the words ‘comprise’, ‘include’ and any variations which may consist of ‘comprising’, ‘comprises’, ‘including’ or ‘includes’ will be understood to imply the inclusion of a stated integer or a group of integers but not the exclusion of any other integer or group of integers. Relative language such as ‘about’ or ‘approximately’ will be understood to be the application of 10% variability in the positive and negative directions of their subject unless stated or specified otherwise. Other definitions for selected terms may be found herein and will apply for the remainder of the section unless redefined otherwise. All other definitions will reflect the common understanding of those ordinarily skilled in the art of the invention or as declared by the subject area.

Features presented through the drawings are referenced using the numerical ordering of the invention stage that they belong to alongside their logical ordering within the drawing itself, with the exception of the first drawing which acts as the initial overview.

Like or the same reference numerals may be used to denote the same or similar features.

The invention will be described in terms of embodiments that relate to analysing a specific portion of articular tissue during orthopaedic surgery to determine the state and classification of that tissue. However, the invention has applicability more generally in the area of intraoperative tissue analysis.

Based on the discussion from the background art herein, it is evident that the current methods used for analysing tissue state and class are suboptimal and only available in limited scenarios. The embodiments of the invention aim to improve this by offering an alternative that allows analysis to occur within an intraoperative environment, increases the accuracy of information derived from this analysis and provides support to involved personnel during related procedures.

This is achieved by utilising an alternative approach that revolves around using various sensors in conjunction with data retrieved and stored across numerous subject analyses. The sensors of differing types produce data based on a single or combination of subjects which can be processed and interpreted to extract information that cannot be procured manually. This is supported by algorithms and methods trained from historically generated data and associated information which can predict state and class information surrounding the subject when given the same input in relation to them.

It should be appreciated that the invention is not limited to orthopaedic operations nor is it limited to any form or type of tissue.

Biological tissue are heterogeneous structures that can absorb, reflect, scatter and re-emit light. The light which is reflected from tissue can be detected and measured through a process called diffuse reflectance spectroscopy. The auto fluorescence light that is emitted can be detected and measured through a process called fluorescence excitation spectroscopy. The majority of tissue, including articular tissue, exhibit unique spectral characteristics in the ultraviolet, visible and near-visible ranges due to their biochemical and morphological states. By sensing and processing these unique spectra, it is possible to classify and determine the state of tissue.

This process typically involves a light source and a method of capture. The light source, whose type is dependent on the form of light being captured, must illuminate the tissue. The method of capture, such as a reflectance probe and spectrometer, will capture the result from the tissue based on this light exposure. It can be used preoperatively on tissue samples and intraoperatively on the patient without resulting in any changes to the state of the tissue. It is mechanically non-invasive as, depending on the light source used, it may be able to reach under the skin to a certain degree, although results received from this are not likely comparable to the exposed equivalent.

Biological tissue mechanically vibrates differently depending on their composition and type. When the tissue acts as the sound source through this vibration, mediums surrounding the tissue such as air and other tissue begin to vibrate as a result. This will continue as sound waves propagate away from the sound source. These sounds waves can be detected and captured through acoustic sensors such as a microphone. By listening to the distinct sound, it is possible to distinguish between different tissue types and determine various aspects of their composition, such as water content or density.

This process requires an initial impact to trigger vibrations in the tissue or sound source. For example, this could be produced by a tightly controlled impact from an actuator, a laser pulse or an air jet. This will create sound waves that can be captured using an acoustic sensor which must be within range of the occurrence and have a high enough sampling rate to draw sufficient detail from. This process can be used preoperatively and intraoperatively, although care must be taken to ensure that the initial trigger impact does not cause damage to the underlying tissue. Depending on the impact trigger, it may be able to reach under the skin to a degree and provide information based on the sound produced whilst taking into account the epithelial tissue acting as a buffer. This will limit the amount of information that can be obtained, although properties such as bone density will still be readily available.

The data retrieved from these different types of sensors typically needs to be parsed, processed and transformed before it can be used in various machine learning, data science and mathematical techniques. This can involve multiple steps depending on the sensors and intended techniques involved. It begins by processing the data which is generally split into two different parts, pre-processing and manipulation. Pre-processing consists of the cleaning, rearranging, formatting and sampling of data to achieve a more usable form. Manipulation consists of scaling or aligning the pre-processed data, decomposing it into its constituent or representative elements and then aggregating the result if necessary.

This is where the training phase begins which builds up a specific machine learning, data science or mathematical technique and allows it to identify its subject. Training consists of matching a set of processed data against their tissue state or classification ground truth and determining which of their values or combination of values result in which truth. Once an adequate amount of data has been channelled through the training phase, the technique can be executed.

Utilising the specific machine learning, data science or mathematical technique once trained usually involves passing it processed data which does not have a matching tissue state or classification ground truth. The values that it contains are run through the technique and are matched against a pre-existing ground truth that was obtained through prior training. This pre-existing truth will be the predicted matching tissue state or classification of the tissue belonging to the data. This process can be repeated using data which does have a matching ground truth to determine the accuracy of a technique depending on how closely the given truth matches its actual one.

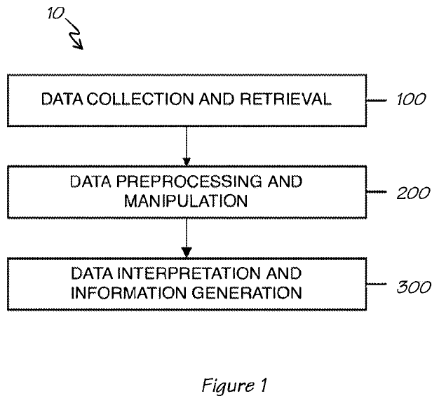

FIG. 1 is a schematic flow diagram depicting the intraoperative tissue state and class analysis method 10, segmented into the individual steps that comprise it. The flow of information between these steps and the individual processing they may contain is explained in overview. Data collection 100 utilises a series of differing sensors in possibly alternating arrangements to produce varying amounts and types of data based on a subject which may be tissue. Data processing 200 pre-processes and manipulates this data to create a form with increased usability and evaluability. Data interpretation 300 passes this processed data into various models and algorithms capable of predicting state and classification information. These models and algorithms are initially trained based on the mapping between historical analysis information and the actual corresponding results and may comprise artificial neural networks (ANNs) or the like as would be appreciated by the skilled addressee. Once populated, new processed data can be passed through wherein identified indicators will be mapped to corresponding values, predicting state and classification information.

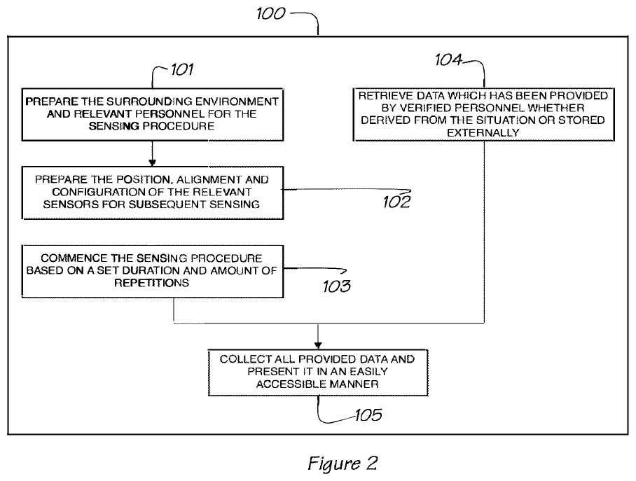

FIG. 2 is a detailed schematic diagram depicting an exemplary data collection and retrieval step 100 of the type depicted in FIG. 1. Data may be collected through a series of sensors that may differ in terms of their type, quantity and arrangement or may be retrieved through external sources related to the subject or the procedure surrounding the subject across a multitude of possible embodiments.

Sensors may act independently or cooperatively as part of a system or collective of sensors where each would cooperate in some fashion to increase the quality or amount of sensed data. Each sensor may be completely self-contained or may require additional devices or systems to handle all or some portion of the required processing.

The physical arrangement of sensors should be made to surround the subject (e.g. a joint e.g. knee, elbow, hip, shoulder etc., of a patient upon which an orthopaedic surgical procedure is being performed or has previously been performed) in a way that maximises the sensing potential of the plurality of sensors whilst resulting in the minimum amount of disturbance to the surrounding environment. If the sensors exist as part of a system in a cooperative setup, then their arrangement should reflect this, for example, sensing the subject from different angles to later stitch the different perspectives together.

The sensors may be automated, manually triggered or controlled through some combination of the two depending on the embodiment. In situations where a proper sensing environment must be created, it would be more opportune to control the sensors manually when this environment is presented. Manual control can be achieved through approaches which may include voice control, gesture control and different forms of physical actuation, the latter of which is present within the preferred embodiment due to its precise control. Otherwise, having them work autonomously to provide information without involvement would be more advantageous. Variations to these approaches may also exist, such as the sensors being triggered automatically once they have perceived the required conditions.

Sensors will typically sense in a periodic fashion as perceived changes may be unlikely to occur constantly, and their rate of sensing may be limited. In some embodiments, sensing may only need to occur once or may be continuous to provide a feed of information in as close to real-time as possible. In situations where snapshots or particular states are sensed, the provision of sensed data in a form of delayed time may be exercised as a number of states may be required to produce gainful data.

The selection of sensors and their configuration will be dependent on their sensing subjects. Sensing a tissue will typically comprise at least one two-dimensional scanner, three-dimensional scanner and hyperspectral or spectral sensors. These should be positioned to surround the tissue with special focus placed on areas of interest, likely relating to the procedure being performed.

Some of these sensors operate in real-time and be periodically sensing. Other sensors may be excluded from direct operation until a point is reached where personnel prepare the theatre environment for ideal sensing conditions prior to reverting the environment after the sensing has occurred. In both cases it would be opportune for trusted personnel to manually trigger the sensors in addition to their autonomous operation. Manual triggers should comprise physical buttons or a touch screen as to allow for efficient, binary interactions.

Turning now to FIG. 2, the data collection and retrieval step 100 of FIG. 1 is described in greater detail. The surrounding environment and relevant personnel should be prepared 101 for any sensing procedures that may occur, depending on the sensors that are in use. This may involve implicit preparation of the environment to ensure or increase the probability of optimal conditions occurring or temporary explicit modification of the environment if the involved sensors cannot sense efficiently during typical conditions. These modifications may comprise having the personnel move any obstructing equipment and adjust any environmental conditions such as lighting. The preferred embodiment will require implicit preparation and some element of explicit preparation. As orthopaedic surgery is generally time-constrained from both a monetary and medical perspective, reliance on periodic sensors that work around the operation is more plausible than those which require constant changes to the setting, although setup configuration changes occurring a small number of times during a procedure may be acceptable.

The configuration of sensors should be prepared 102 for any sensing procedures that may occur, provided their environment is in such a way that allows this to be possible or at least efficient. This may involve changing the position, alignment and orientation of sensors both independently and in relation to each other. Additional equipment such as stands or platforms may be necessary for these changes. In the preferred embodiment, the sensors will already have the required configuration as part of a pre-constructed system or platform. When the opportunity arises, this system as a whole can be moved into place in a relatively small time-frame, reducing environmental impact and disruption. After preparation 101 & 102 is completed, the sensing procedure 103 can begin.

Sensing will occur based on a set duration which determines the number of repetitions possible based on the particular types and quantities of sensors present in the particular setup arrangement. In embodiments that require the environment and sensor configuration to be adjusted for sensing, these parameters are likely to be constrained by their setting. During orthopaedic surgery, this duration is likely to be only be a few minutes long as time is crucial for success of the surgical procedure, with the number of sensing repetitions being similarly limited as a result. In embodiments that allow for passive sensors, the duration may be dependent on the total lifetime of the subject being sensed or the actions performed in relation to it, allowing for a comparatively higher number of repetitions with respect to the capabilities of the involved sensors. After sensing has occurred, the preparatory measures 101, 102 implemented prior may be reverted if necessary.

Data may also be collected directly through provisions from verified personnel or systems 104 which may include documents, records and databases. In particular embodiments, this would comprise any resources that could provide additional information on the patient or the operation that they are undergoing, such as patient records, medical records and historical operation or surgery data.

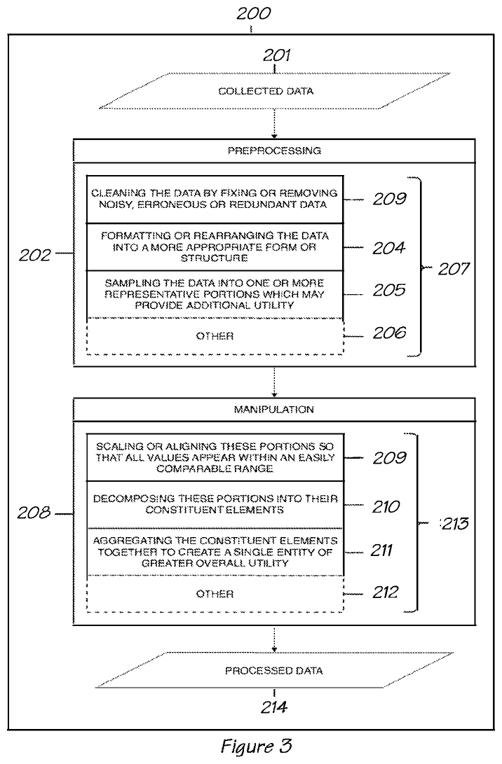

All data sensed and provided will be collected and presented in an easily accessible manner 105 as required by the necessary processing step 200, depicted in detail in FIG. 3. Collection may involve the extraction of data in whichever format is deemed the most usable, generally determined by the sensor it originated from. Sensed data may initially appear in a raw form which must be converted into data in which meaning can be drawn from, typically by an external control unit. Similarly, provided data may appear in a form that cannot be easily accessed, such as paper, which requires input into a digital system to rectify. In particular embodiments, all data would be stored in the same way so that they may be accessed in the same way. This method of storage would ideally be the random-access memory (RAM) of a central system, although a solid-state drive or hard disk may be used instead depending on the raw amount of data and processing speed required for traversal. In other embodiments, a database may be used to store and access this data. It may use strict storage and access guidelines as imposed by SQL or be more flexible and scalable using technology such as NoSQL.

FIG. 3 is a detailed schematic diagram depicting an exemplary data pre-processing and manipulation step 200 of the type depicted in FIG. 1. Data will be processed to transform it into a form of greater utility, generally in terms of both its usability and evaluability.

The data source 201 comprising collected data involved wherein the collected data comprises, at least, the raw sensed data and/or the data provided from trusted personnel or systems, which were either generated from the plurality of sensors located about the subject and/or provided externally. This data should contain enough information to determine the state and class of its associated tissue.

The intent behind processing the data is to best prepare it for training and execution within predictive algorithms and methods including for example machine-learning or artificial neural network systems as would be appreciated by the skilled addressee. This may involve different types of pre-processing and manipulation to transform the data into a form that produces the most benefit with respect to this usage.

Pre-processing the data involves transforming it into a form of superior usability 202 in preparation for and to produce the most utility from subsequent data manipulation. This data may initially be in a somewhat raw form which may contain noise, errors or redundancy. If data containing these flaws is used during normal processing, redundant calculations, inconsistencies or incorrect results may occur. These must therefore be fixed or removed 203 depending on their type and severity.

Noisy data may be defined as data that is partially correct but contains other portions that are corrupt or in error. The proportion between the correct data and that which is in error is an indicator about the type of actions that can be taken in response to it. If only a small amount is in error, then it may be possible to fix this amount based on the correct data, or it could be removed provided that the remaining data provides sufficient benefit in its reduced form. If the amount of incorrect data is large however, then removing the data as a whole is likely the only option.

Erroneous data may be defined as data that is wrong and contains values that cannot possibility exist either through the medium that created it or in relation to surrounding data. Erroneous data cannot be fixed in most scenarios as it typically has no relation to the value that it should have been and is therefore usually removed.

Redundant data may be defined as data which, although not in error, does not add any additional value or benefit to the data set as a whole and only serves to increase its volume and introduce inconsistencies. Redundant data cannot be fixed as it is technically correct and is therefore usually removed or ignored instead.

Removing or fixing data is highly dependent on the origin and format of the data and the severity of the portions in question. Removal is relatively straightforward depending on the format but will leave the remaining data in a reduced state. The data will remain valid in some cases, but others may require additional modifications to achieve this. This may entail combining the remaining data with other sets of reduced data to create complete sets or replacing the data with dummy data that will not affect the end result. The fixing of data in comparison is more difficult and requires knowledge about the expected structure to determine what is missing or wrong so that it can be rectified. Techniques to achieve this are highly dependent on the data itself and may not even be possible. In the preferred embodiment, all redundancy and errors will be directly removed, whist any noise will be fixed if additional benefit can be discerned.

Collected data 201 will typically need to be rearranged and formatted to increase access efficiency and make its storage more logical in terms of processing 204. This is because its initial form will likely be based on the ordering and format of its origin, such as a specific sensor, system or set of personnel, which is suboptimal for manipulation.

In the preferred embodiment, and specifically for the intended predictive approaches, rearrangement will consist of grouping together data which may have established similarities or other relations. This makes accessing or searching for related data or data which cleanly represents a particular aspect or series of aspects easier and more efficient. Formatting will consist of structuring these different groupings in ways that allow different sets of data to be manipulated and analysed simultaneously and subsequently. This will make traversal from one set of data to another related set of data relatively simplistic and computationally inexpensive. Other embodiments will have differing approaches to formatting and arrangement depending on the type of manipulation and subsequent predictive approaches intended for the data.

Collected data 201 may be sampled 205 as part of pre-processing to create different portions which may provide additional utility as opposed to operating based on the data as a whole. Sampling consists of reducing the data pool into one that is more advantageous towards a specific type of usage, such as the data as a whole being reduced to only parts that may be deemed as representative.

In the preferred embodiment, and specifically for the intended predictive approaches, the data will initially be sampled to create a single data pool that is more representative than the data as a whole. This is to say that the utility provided by this representative data pool should be equal to or superior than it was originally. This representative pool will then be split into three different segments. The first and largest segment, known as the training set, will be used for training the predictive algorithms and methods. The second smaller segment, known as the test set, will be used for testing trained predictive approaches. The third smaller segment, known as the validation set, will be used for validating the results of trained predictive approaches that have produced favourable accuracy against the test set.

Other embodiments will likely use a similar sampling approach as is consistent with predictive approaches, although additional customisations may be made depending on their specifics.

Manipulating the data 208 involves transforming it into a form of superior evaluability in preparation for and to produce the most utility from subsequent predictive algorithms or methods. This data may initially be in a form wherein each value exists based on how it was expressed originally. Since each expression will likely be different across the data, achieving an appropriate level of comparability between the different sets may not be viable or may be done so to suboptimal degrees. By scaling or aligning these values to a common point 209, comparability between the different sets increases.

In the preferred embodiment, and specifically for the intended predictive approaches, all values existing within data sets that may be deemed comparable and which have direct or similarly equivalent initial expressions should be scaled. This is because some types of predictive analysis generally work better when all data exists within some known range. It also makes handling the data and distinguishing it easier, especially when presenting the data, should the need arise. Other embodiments will likely use similar scaling techniques which will again be based on their intended predictive algorithms or methods.

Collected data 201 may be reduced, split or decomposed into their constituent elements 210 as part of data manipulation. These individual elements compose the data and can be used to identify which existing features may be more beneficial or representative in comparison to others. This is important for predictive analysis as these types of features generally make good indicators, which may greatly increase their utility.

In the preferred embodiment, and specifically for the intended predictive approaches, data will be decomposed into constituent elements if it can be seen that the individual elements or otherwise features make a considerable contribution in determining the overall description of the data as a whole.

Collected data 201 and the constituent elements derived from it may be aggregated together into a single entity 211 as part of data manipulation. The aggregated entity should provide more utility in comparison to the individual elements or data which were used to create it, although this may not be the case if the decision was made from a storage or computational perspective.

Aggregation approaches are largely dependent on the application, the type and expression of data, and the forms of processing their results will be used with. Simplistic approaches may involve averaging the involved data together whilst more complex ones may involve providing a weighting to each individual element and performing a procedure that processes and combines them based on these weightings. As the amount of information relating to the context of the data and application increases, the complexity and utility granted by these aggregation approaches may do so as well.

In the preferred embodiment, and specifically for the intended predictive approaches, elements should be aggregated together if additional utility will be produced. This means that if an aggregated entity indicates the properties of a set of data better in comparison to the individual elements, then the aggregation should be maintained. It may be performed for all sources of data but will likely be restricted to data of similar origin as different aggregation algorithms may require some amount of similarity to be productive. Other embodiments will likely aggregate data in a similar manner, with their dependencies determining how and to what degree this will occur.

The final processed data 214 will be produced after the collected data 201 has been pre-processed and manipulated. Other pre-processing approaches 206 and manipulation approaches 212 may exist in addition to those mentioned above as would be readily appreciated by the skilled addressee. The ordering and existence of these pre-processing approaches 207 and manipulation approaches 213 may not necessarily reflect the ordering and existence of the approaches herein.

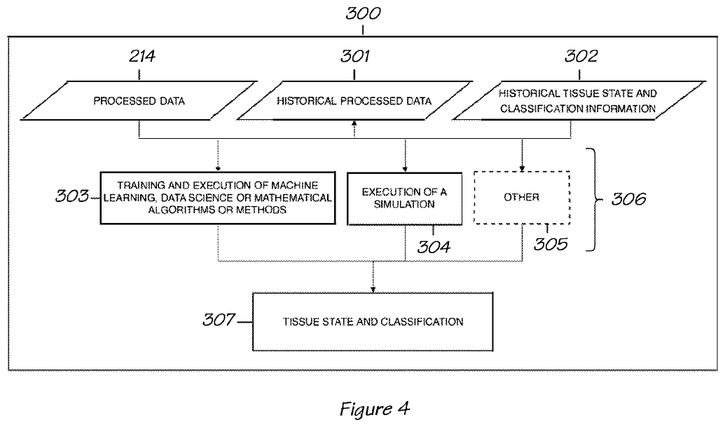

FIG. 4 is a detailed schematic diagram depicting an exemplary data interpretation and information generation step 300 of the type depicted in FIG. 1. This involves using two different data sources within a series of different predictive approaches to generate information and values which may provide insight into the state and classification of a particular tissue.

The first data source 214 is the data recently processed (refer to FIG. 3) to produce additional utility during predictive analysis as detailed in FIG. 3. The second data source is the same except that it contains historical processed data 301 that has been generated prior and retrieved 104. These sources will be used as derivable data where indicators and other mapping mechanisms may be found.

The third data source 302, which contains a specific set of values corresponding to each value within the second data source, is the historical data of actual tissue state and classification information. This is used as the ground truth and is what may be predicted.

Predictions may be generated based on the first data source 214 by training and executing different forms of machine learning, data science and mathematical algorithms or methods 303. In the preferred embodiment, this will mainly consist of different supervised approaches. These types of approaches generally operate in two different phases comprising the training phase and the execution phase.

Turning now to FIG. 4 the data interpretation and information generation step 300 of FIG. 1 is described in greater detail. Process 300 includes training of the algorithms for the data interpretation and output in a machine learning or ANN framework. The training phase involves the second and third data sources including the processed data 214 generated by the plurality of sensors located about the subject, and the retrieved historical data 104 wherein each set of data in the second data source 301 maps to a specific set of values in the third 302. It consists of identifying indicators within each set of data that are either partially or majorly responsible for this mapping such that if another set of data contained these same indicators, it would be likely that it would also have the same or similar corresponding values. This will continue until a mapping structure has been developed that will map parsed indicators to values that they most commonly refer to.

The training phase is often performed using different segments of data as opposed to the data as a whole which may include training, testing and validation segments, where the data and corresponding values are known for each. It will initially begin by generating a mapping structure corresponding to only the training segment. The data within the test segment will then be ran through this structure, with the values it returns being compared to the actual known values of the segment. This will provide a measure of accuracy depending on how close the returned values are to the actual ones. If this accuracy is satisfactory (somewhere between 95-100% based on the preferred embodiment) then it is tested again using the validation segment. This is to simulate its performance on real world data as although it has seen the training and test segments previously, the validation segment will remain unknown to it. This ensures that the mapping structure will perform well on all data as opposed to only the test segment, a phenomenon known as over-fitting.

The execution phase involves only the first data source 214 which has no known corresponding values. It initially consists of identifying the same indicators found during the training phase within each set of data in this source. These indicators are then given to the previously created mapping structure to identify the values that they correspond to.

Supervised algorithms or methods differ greatly in their complexity as well as their predictive power and using various types of them concurrently may produce beneficial results in addition to a point of comparison. These algorithms or methods may include linear and polynomial regression, logistic regression, naive bayesian networks, bayesian networks, support vector machines, decision trees, random forests, k-nearest neighbour classifiers and neural networks.

Other embodiments may use different predictive approaches, including unsupervised, semi-supervised and reinforcement approaches. Unsupervised and semi-supervised algorithms or methods are provided a data set and are made to extract meaning from it without any or with little direction as to what it is that they are looking for. This allows unknown information or connections existing within the data to be found which may provide additional utility depending on what they are and their consistency in other data sets.

Reinforcement algorithms or methods may attempt to run a series of calculations with the goal of producing a particular value. They are provided positive or negative stimulus depending on the accuracy of this value in comparison to what it should have been. When provided positive stimulus, they will continue performing the same calculations that they have done and may perform additional ones which are similar to these. When provided negative stimulus they may stop performing their current calculations and try some that are different to varying degrees. A degree of randomness is typically added to these algorithms to give them a starting point, which means that they may require more execution cycles to reach a satisfactory result in comparison to the prior predictive analysis approaches.

Predictions may be generated based on processed data 214 by running a simulation 304 that involves the different types of scenarios and conditions which may affect the state of the tissue. These types of instances will likely be simulated mathematically with probabilistic measures added to account for situations that are not currently determinative.

In the preferred embodiment, the simulation will be designed around the different procedures or techniques which may occur intraoperatively and their associated effects on the tissue. It will be provided two main sources of data.

The first source will be processed data 214 that contains various information relating to the state and class of the tissue. This will be used as a foundation platform for the simulation to begin its processing.

The second source 301 will be information similar to the first but having occurred after a particular event or procedure where any associated variables are known. This information will indicate the effect that these variables may have participated in creating.

Currently the simulation has been referred to as singular, but this may not be the case if additional benefit can be found by having multiple different simulations or by dividing a single simulation into multiple individual simulations that each have their own purpose or predictive goal. Considering the complexity that is usually involved, division may be advantageous at least from a development and production point of view.

Other embodiments may utilise different simulations depending on their context and application. This would likely be dependent on their subject and the desired results of the simulation.

Predictions generated will be used to provide insight into information relating to the state and classification of tissue 307. These two sets of identifying information comprise various properties and characteristics which generate their value as a whole. They can be used individually or together to determine and inform the results of various procedures or events surrounding specific tissue.

Tissue state comprises a series of descriptors that provide information relating to tissue health. These descriptors generally revolve around a particular aspect of the tissue and may change over time or after significant events. Composition is one such descriptor that describes the types of minerals that may comprise the tissue. The different minerals and their abundances generally make a reliable indicator as to the health and age of a specific tissue and notably differ when variations to these properties are present. This is especially useful when gauging tissue density, which is defined by how tightly packed these minerals, or at least specific minerals, are in relation to each other. Heat consistency is another such descriptor which describes the temperature of the tissue and how this is distributed across it. Measuring heat consistency is often a good way to monitor how the tissue is being affected when performing operations such as osteotomies. Other state descriptors may include hydration which may describe the water content existing within the tissue that can be useful in measuring the impact of any prior osteotomies; necrosis which describes the death of tissue cells which may have been caused by the method of osteotomy or internal issues within the body; colouration which describes the particular colour that the tissue exhibits wherein any variations typically cannot be discerned without advanced visual sensors; and reflectance which describes how much and what colours the tissue can actively reflect. Tissue state information or descriptors may be used individually, within a selective set or as a whole to determine or predict the results that a particular procedure or set of variables has on the tissue.

Tissue classification comprises defining the type of a particular tissue and any specialisations that can be derived on top of that. This may comprise bone, cartilage, fat, ligament, muscle and meniscus. It may be used to define the type of tissue either individually or as part of a selected area or region and can reinforce or further inform conclusions made in conjunction with state information.

Other predictive approaches and resultant information may exist external to those explicitly outlined herein 305. Predictive approaches may not necessarily only be executed singularly, they may also be executed concurrently and sequentially if reason exists to do so 306.

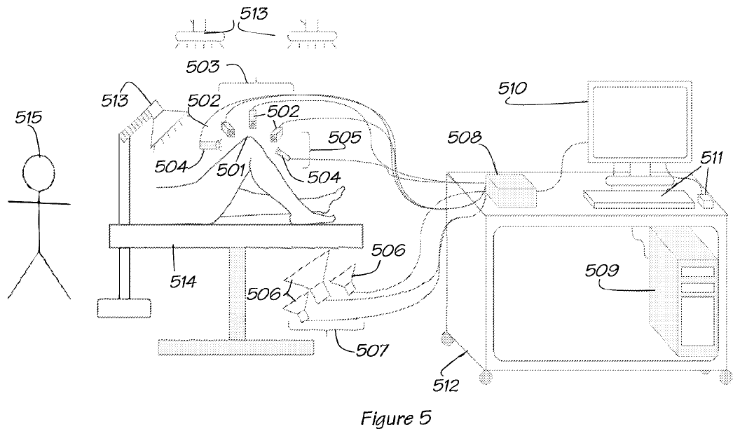

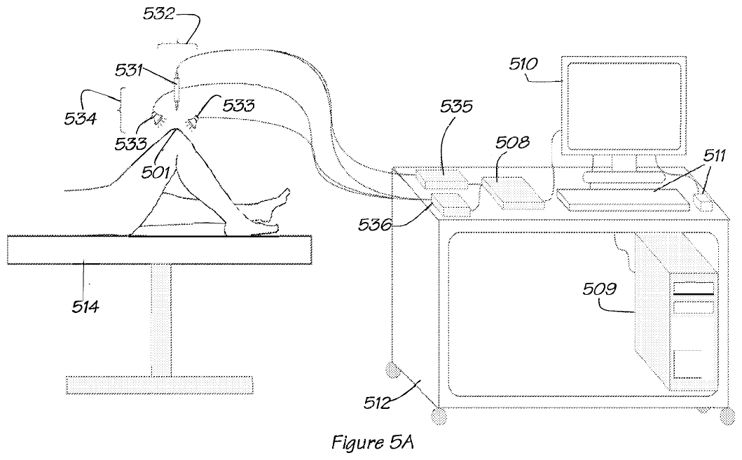

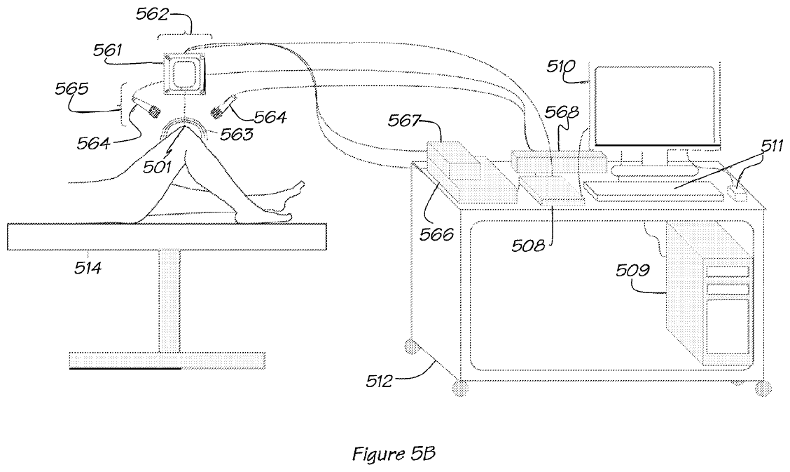

FIG. 5 illustrates generally a basic embodiment for tissue state and class analysis and the intraoperative environment in which it is used. It includes a plurality of sensors 502 arranged about the subject joint 501 of the patient (in the illustrated case, the patient's left knee). The plurality of sensors 502 may comprise different types and models and not necessarily have uniformity. The number and arrangement 503 of these sensors may differ depending on the subject 501 and the particularities of the application in analysing a particular subject.

Some sensors may not work independently and may instead require additional sensors or assistant devices 504 such as, for example, light sources. These devices may comprise different types and models and may also differ in their number and arrangement 505 in accordance with requirements.

Sensors 502 may not generate data directly and may instead rely on a separate capture device 506 to handle or support this generation process to varying degrees. These capture devices 506 may comprise different types and models and may also differ in their number and arrangement 507 accordance with requirements, with these specifics likely being dependent on their associated sensors 502.

In particular embodiments, the subject of these sensors and any additional equipment will be articular tissue derived from a knee 501 during orthopaedic surgery e.g. different joints (hip, elbow, shoulder etc.) of the patient upon which an orthopaedic procedure is being performed. This may however be any other form of tissue existing within any other form of surgery. The illustrated arrangement provides an example as to how the sensors and associated equipment may be positioned relative to the subject 501 in the present embodiment. By positioning the sensors around the subject 501 through many different angles, it can be assumed that a high level of visibility may be achieved.

Temporary or permanent adjustments or modifications to the intraoperative environment during associated medical procedures or surgeries may be necessary to achieve plausible sensing conditions or increased sensing accuracy. This may comprise adjustments to immediate environmental conditions such as lighting 513, changes to the position or arrangement of the subject (e.g. by movement of the surgical bed/table 514 relative to the sensors 502), and the removal or repositioning of personnel 515 within the environment.

Similar adjustments or modifications may need to be made for the sensors 502 and their associated equipment such as for example, computers 509 and monitors 510, controllers 508 etc. Depending on the type of medical procedure or surgery, this associated equipment may need to be outside of the operating environment completely and reintroduced at specific intervals when it is deemed necessary or safe for the patient. In particular embodiments, the associated equipment will be positioned safely around the subject as to not disturb or hamper the vision of related surgical or support personnel. Once a point in the surgery has been reached or their involvement is deemed necessary, the associated equipment may be moved closer or to a more opportune location prior to being moved back. Other embodiments may not require any repositioning, with the sensors existing simply as passive devices or they may take precedence such that the personnel will be moved into position at points determined by the system with the sensors given priority otherwise.

All data and information provided by the sensors 502 is advantageously received by a peripheral controller 508. This controller 508 may perform any amount of processing required to compile the data into a form that may be delivered to and interpreted by the remainder of the system 509.

The collected data will be processed within the system 509 responsible for executing the analysis process. As defined through the processes depicted in FIGS. 1 to 4, this processing will comprise pre-processing and manipulating the data, generating a means of predictive analysis based on this data and executing this analysis to derive meaningful results.

All generated information will be displayed through a form of communicative medium that intended personnel can interpret. In the preferred embodiment, this will be an LCD screen, such as that provided by a computer monitor 510. In other embodiments, this may comprise sound recordings, representative lighting or printed material.

Interaction may be possible with the selected communicative medium based on actions provided by the user. In the preferred embodiment, this will comprise a keyboard and mouse 511 which will enable the LCD screen 510 to be interacted with in a natural and familiar way. It may also allow the forms of possible interaction to be more complex with a larger range of options. In other embodiments, this may comprise voice commands, gesture commands and a touch screen interface.

System components may be arranged together within a singular system or container 512 such that all interactions with the individual components will comprise an interaction with this container. In the preferred embodiment, a structure housing all of these components will be developed such that they may be moved, adjusted and associated with each other in a clear and simplistic manner. In other embodiments, they may be individually positioned and arranged such that each exists independently and can be adjusted without necessarily effecting the state of the remaining components.

FIG. 5A illustrates generally an implementation of the basic embodiment of FIG. 5 which utilises spectral or hyperspectral sensors for intraoperative tissue state and class analysis. It includes an array of spectral or hyperspectral sensors 531 which may comprise different types and models and not necessarily have uniformity. They may have any singular or combination of bands available within the electromagnetic spectrum, with this likely being determined by the bands that evoke the greatest response from their subject. The number and arrangement of these spectral sensors may differ 532 depending on their subject and application. In the preferred embodiment, each subject will have a single corresponding spectral sensor unless benefit exists in having multiple, such as in scenarios where multiple bands are required but cannot be contained within a single sensor.

Spectral or hyperspectral sensors generally require a specific type or level of light to produce useful information. Although this light may be achieved from natural lighting or from those introduced by the surgical environment, specialised sources that can be controlled or which exhibit a specific form of light may be necessary. These light sources 533 typically consist of a generator 536, a transmission medium which is usually a type of fibre, and an emitter device such as an LED or some other form of director. They may comprise UV, visible and infrared lights of variable brightness and strength and may differ in type, model, number and arrangement 534. They may exist independently or be coupled within other hardware such as a spectral or hyperspectral sensor. In the preferred embodiment, each spectral or hyperspectral sensor will have as many light sources as necessary to illuminate their subject and increase their accuracy.

Temporary adjustments to the environment may be necessary to allow the spectral or hyperspectral sensors to perform as intended. Since they operate based on light, this will typically comprise adjustments to the lighting which may involve dimming external lights to increase the effectiveness of specialised lights, removing any obstacles in their vision or of which may cause shadows and decreasing the distance between them and the subject to encourage light penetration.

Light sensed by spectral or hyperspectral sensors may need to be passed through a spectrometer 535 before it can be utilised. This is a device that takes light and splits it into an array of separate colours determined by the possible available bands. Each spectrometer will be matched with at least one spectral or hyperspectral sensor with the nature of this correspondence being dependent on the type and model of all involved components.

The light sources and spectrometers will be controlled by and communicate with single or multiple peripheral controllers 508 depending on the application and sensor arrangement. This controller will likely perform some form of preliminary processing to transform the received data into a more usable form prior to passing it to the remainder of the system 509.

The preferred embodiment will likely possess some form of spectral or hyperspectral sensing in order to distinguish between the different types of tissue as each one may reflect and absorb differing amounts and frequencies of light. These amounts may also be influenced by variables relating to the state such as hydration and necrosis which may also be discerned through these sensors.

FIG. 5B illustrates generally an implementation of the basic embodiment of FIG. 5 which utilises acoustic sensors for intraoperative tissue state and class analysis. It includes an array of acoustic sensors 564 which may comprise different types and models and not necessarily have uniformity. They may have any possible sampling rate, with this likely being determined by the sound evoked from physically impacting their subject. The number and arrangement of these acoustic sensors may differ 565 depending on their subject and application. In the preferred embodiment, each subject will be surrounded by multiple acoustic sensors to sample the sound produced from many different angles.

Acoustic sensors can only perceive sound from a static tissue if it is impacted by something 563. The actuator 561 of this impact must therefore produce it in a constant and measured fashion as if the amount of force or any other variables differ, a different sound will be produced. Although the signature of this sound will likely be the same, having an actuator that will reduce any inconsistencies will be advantageous. The number and arrangement of these actuators may differ 562 depending on the application as creating sound from multiple different angles provided that the actuators are appropriately synchronised may be beneficial. In the preferred embodiment, as illustrated, the actuator will comprise a single laser system that will emit a measured and consistent single pulse of light into the tissue to produce sound. By using a highly controlled actuator, the resulting sound frequencies will be more consistent. This is important when dealing with subjects whose potentially differing states may produce subtly similar sounds such that even relatively small amounts of actuator variance may affect the final result.

Temporary adjustments to the environment may be necessary to allow the acoustic sensors to perform as intended. Since they operate based on sound, moving the sensors closer to the source of this sound will typically give that specific audio frequency more prevalence in comparison to other sounds produced in the environment. However, this also means that data returned from these sensors will likely vary based on their arrangement, although will still maintain the same or a similar signature. This makes it likely that large movements of the sensors would provide little benefit in comparison to placing them statically. In the preferred embodiment, the sensors will be placed statically at the beginning of a medical procedure or surgery and remain there until they are no longer needed. Their development will therefore likely focus on identifying signatures instead of raw acoustic signals.

The actuator 561 or actuators used will require single or multiple devices to control their output and triggering. The illustrated laser system will require two different devices, with the configuration and selection of them being dependent on the strength and type of laser. The first of these is the laser current source 566 which generates the required current internally prior to sending it to the laser. The second is the laser chiller 567 which will cool the laser between pulses to ensure that it does not overheat and damage itself or its surroundings. The preferred embodiment will require both of these components.

Acoustic signals monitored by the sensors will need to be received and transformed into a form that can be interpreted which is typically handled by an audio interface or receiver 568. The configuration of the different sensors including their rate and sensitivity can be controlled through this system. The preferred embodiment will use a single receiver with all involved acoustic sensors being attached to it such that their signals may be interpreted as a whole instead of individually as they will likely perceive the same subject but from different angles and positions.

All components may be attached to single or multiple peripheral controllers 508 depending on the application and sensor arrangement. This controller will likely perform some form of preliminary processing to transform the received data into a more usable form prior to passing it to the remainder of the system 509.

The preferred embodiment will likely possess some form of acoustic sensing m order to determine specific types of tissue state information such as density and hydration. The type of tissue can then be derived from this or used as reinforcement for conclusions developed through other sensing methods.

The features presented herein may be performed electronically through any capable system or machine that can complete them within any restrictions applied by their particular application. This may be performed online, offline or in a capacity that relies on some combination of the two.

Data extracted or generated as a result of the features presented herein may be stored electronically which can be done offline, online, or through some combination of the two. This may be accessed immediately or in a delayed time frame for retrieval, processing and any other form of usage. All types of data may be stored and may be maintained singularly, intermittently, routinely or through any other timing paradigm.

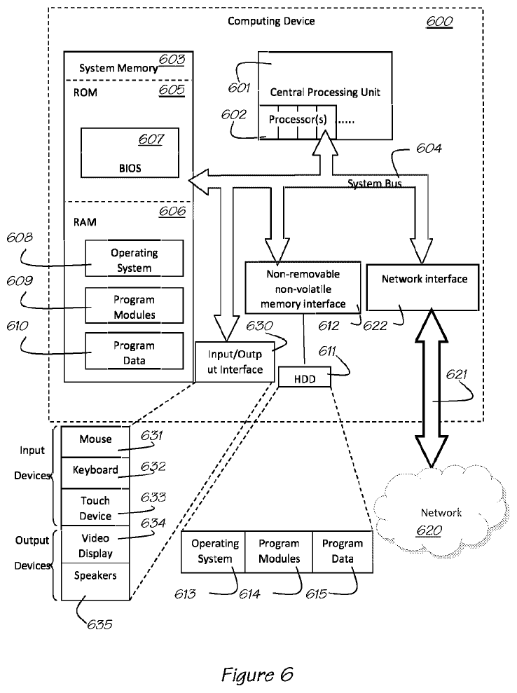

The method 10 of biological state analysis and classification of bone and soft tissue in orthopaedic applications depicted in FIG. 1 and associated sub methods (e.g. methods, 100, 200, and 300 depicted in FIGS. 2 to 4) may be implemented using a computing device/computer system 600, such as that shown in FIG. 6 wherein the processes of FIGS. 1 to 4 may be implemented as software, such as one or more application programs executable within the computing device 600. In particular, the steps of methods 10, 100, 200, and 300 depicted in FIGS. 1 to 4 are effected by instructions in the software that are carried out within the computer system 600. The instructions may be formed as one or more code modules, each for performing one or more particular tasks. The software may also be divided into two separate parts, in which a first part and the corresponding code modules performs the described methods and a second part and the corresponding code modules manage a user interface between the first part and the user. The software may be stored in a computer readable medium, including the storage devices described below, for example. The software is loaded into the computer system 600 from the computer readable medium, and then executed by the computer system 600. A computer readable medium having such software or computer program recorded on it is a computer program product. The use of the computer program product in the computer system 600 preferably effects an advantageous apparatus for the biological state analysis and classification of bone and soft tissue in orthopaedic surgery applications including post-operative care and evaluation of orthopaedic surgical procedures.

With reference to FIG. 6, an exemplary computing device 600 is illustrated. The exemplary computing device 600 can include, but is not limited to, one or more central processing units (CPUs) 601 comprising one or more processors 602, a system memory 603, and a system bus 604 that couples various system components including the system memory 603 to the processing unit 601. The system bus 604 may be any of several types of bus structures including a memory bus or memory controller, a peripheral bus, and a local bus using any of a variety of bus architectures.

The computing device 600 also typically includes computer readable media, which can include any available media that can be accessed by computing device 600 and includes both volatile and non-volatile media and removable and non-removable media. By way of example, and not limitation, computer readable media may comprise computer storage media and communication media. Computer storage media includes media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by the computing device 600. Communication media typically embodies computer readable instructions, data structures, program modules or other data in a modulated data signal such as a carrier wave or other transport mechanism and includes any information delivery media. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 603 includes computer storage media in the form of volatile and/or non-volatile memory such as read only memory (ROM) 605 and random-access memory (RAM) 606. A basic input/output system 607 (BIOS), containing the basic routines that help to transfer information between elements within computing device 600, such as during start-up, is typically stored in ROM 605. RAM 606—typically contains data and/or program modules that are immediately accessible to and/or presently being operated on by processing unit 601. By way of example, and not limitation, FIG. 6 illustrates an operating system 608, other program modules 609, and program data 610.

The computing device 600 may also include other removable/non-removable, volatile/non-volatile computer storage media. By way of example only, FIG. 6 illustrates a hard disk drive 611 that reads from or writes to non-removable, non-volatile magnetic media. Other removable/non-removable, volatile/non-volatile computer storage media that can be used with the exemplary computing device include, but are not limited to, magnetic tape cassettes, flash memory cards, digital versatile disks, digital video tape, solid state RAM, solid state ROM, and the like. The hard disk drive 611 is typically connected to the system bus 604 through a non-removable memory interface such as interface 612.

The drives and their associated computer storage media discussed above and illustrated in FIG. 6, provide storage of computer readable instructions, data structures, program modules and other data for the computing device 600. In FIG. 6, for example, hard disk drive 611 is illustrated as storing an operating system 613, other program modules 614, and program data 615. Note that these components can either be the same as or different from operating system 608, other program modules 609 and program data 610. Operating system 613, other program modules 614 and program data 615 are given different numbers hereto illustrate that, at a minimum, they are different copies.

The computing device also includes one or more input/output (I/O) interfaces 630 connected to the system bus 604 including an audio-video interface that couples to output devices including one or more of a video display 634 and loudspeakers 635. Input/output interface(s) 630 also couple(s) to one or more input devices including, for example a mouse 631, keyboard 632 or touch sensitive device 633 such as for example a smartphone or tablet device.

Of relevance to the descriptions below, the computing device 600 may operate in a networked environment using logical connections to one or more remote computers. For simplicity of illustration, the computing device 600 is shown in FIG. 6 to be connected to a network 620 that is not limited to any particular network or networking protocols, but which may include, for example Ethernet, Bluetooth or IEEE 802.X wireless protocols. The logical connection depicted in FIG. 6 is a general network connection 621 that can be a local area network (LAN), a wide area network (WAN) or other network, for example, the internet. The computing device 600 is connected to the general network connection 621 through a network interface or adapter 622 which is, in turn, connected to the system bus 604. In a networked environment, program modules depicted relative to the computing device 600, or portions or peripherals thereof, may be stored in the memory of one or more other computing devices that are communicatively coupled to the computing device 600 through the general network connection 621. It will be appreciated that the network connections shown are exemplary and other means of establishing a communications link between computing devices may be used.

It should be understood that the features presented herein and the different processes that they contain do not necessarily need to be performed in the described order nor do they require a specific environment or situation. Their ordering, nature, preparation and execution may be dependent on numerous circumstances as is typically the case with medically applicable inventions or methods.

It will be appreciated by those skilled in the art that variations and modifications to the invention described herein will be apparent without departing from the spirit and scope thereof. The variations and modifications as would be apparent to persons skilled in the art are deemed to fall within the broad scope and ambit of the invention as herein set forth.

Future patent applications may be filed in Australia or overseas on the basis of, or claiming priority from, the present application. It is to be understood that the following provisional claims are provided by way of example only and are not intended to limit the scope of what may be claimed in any such future application. Features may be added to or omitted from the provisional claims at a later date so as to further define or re-define the invention or inventions.

1-67. (canceled)

68. A method for intraoperative analysis of biological tissue, the method comprising:
directing a controlled laser pulse to a region of the biological tissue, wherein the controlled laser pulse physically interacts with and structurally modifies the region of the biological tissue;
detecting, using at least one acoustic sensor, an acoustic response generated by the biological tissue as a result of the controlled laser pulse;
processing data representative of the acoustic response to identify one or more characteristics of the acoustic response; and
determining at least one tissue characteristic of the region of the biological tissue based at least in part on the one or more characteristics of the acoustic response generated in response to the controlled laser pulse.

69. The method of claim 68, wherein the controlled laser pulse changes a form of at least a portion of the region of the biological tissue.

70. The method of claim 68, wherein the at least one tissue characteristic comprises at least one of composition, hydration, density, necrosis, coloration, reflectance, or heat consistency.

71. The method of claim 68, wherein determining the at least one tissue characteristic comprises determining a tissue classification including at least one of cancellous bone, cortical bone, cartilage, fat, ligament, muscle, or meniscus.

72. The method of claim 68, wherein the acoustic response is detected using a plurality of acoustic sensors positioned at different respective positions or angles relative to the region of the biological tissue.

73. The method of claim 68, wherein the controlled laser pulse is generated by a laser system configured to provide a measured and consistent laser output to the biological tissue.

74. The method of claim 68, wherein the one or more characteristics of the acoustic response comprise one or more frequencies, amplitudes, measurements, or identifiable features of the acoustic response.

75. The method of claim 68, wherein determining the at least one tissue characteristic comprises applying, to the processed data representative of the acoustic response, one or more of a trained machine learning model, a data science model, a mathematical model, or a computational model.

76. The method of claim 68, wherein the detecting, processing and determining are performed intraoperatively in real-time or near-real-time.

77. A method for intraoperative analysis of biological tissue, the method comprising:
directing a controlled laser pulse to a region of the biological tissue, wherein the controlled laser pulse structurally modifies the region and causes a change in form of at least a portion of the region;
sensing, using at least one three-dimensional sensing modality, data representative of the change in form resulting from the structural modification;
processing the sensed data to identify one or more physical characteristics of the structurally modified region; and
determining at least one tissue characteristic of the biological tissue based at least in part on the one or more physical characteristics resulting from the structural modification caused by the controlled laser pulse.

78. The method of claim 77, wherein the at least one three-dimensional sensing modality comprises one or more of three-dimensional metrology, optical coherence tomography, or a three-dimensional scanner.

79. The method of claim 77, wherein the sensing is performed during or following the structural modification of the region.

80. The method of claim 77, wherein sensing the structurally modified region comprises sensing the region from a plurality of different angles or perspectives.

81. The method of claim 77, wherein the at least one tissue characteristic comprises at least one of composition, hydration, density, necrosis, coloration, reflectance, or heat consistency.

82. The method of claim 77, wherein determining the at least one tissue characteristic comprises applying, to the processed sensed data representative of the structurally modified region, one or more of a trained machine learning model, a data science model, a mathematical model, or a computational model.

83. The method of claim 77, wherein the controlled laser pulse is generated by a laser system configured to provide a measured and consistent laser output to the biological tissue.

84. A system for intraoperative analysis of biological tissue, the system comprising:
a laser system configured to direct a controlled laser pulse to a region of the biological tissue, wherein the controlled laser pulse is configured to physically interact with and structurally modify the region of the biological tissue;
at least one acoustic sensor configured to detect an acoustic response generated by the biological tissue as a result of the controlled laser pulse; and
at least one processor configured to:
receive data representative of the acoustic response;
process the data representative of the acoustic response to identify one or more characteristics of the acoustic response; and
determine at least one tissue characteristic of the region of the biological tissue based at least in part on the one or more characteristics of the acoustic response generated in response to the controlled laser pulse.

85. The system of claim 84, wherein the laser system is configured to provide a measured and consistent laser output to the region of the biological tissue.

86. The system of claim 84, wherein the at least one acoustic sensor comprises a plurality of acoustic sensors arranged at different respective positions or angles relative to the region of the biological tissue.

87. The system of claim 84, wherein the at least one processor is configured to determine, based on the processed data representative of the acoustic response, at least one of tissue type, composition, density, or hydration.
